(19) United States
(12) Patent Application Publication
(10) Pub. No.: US 2014/0024940 A1
(43) Pub. Date: Jan. 23, 2014

(54) ULTRASONIC DIAGNOSTIC APPARATUS AND SENSOR SELECTION APPARATUS

(71) Applicants: TOSHIBA MEDICAL SYSTEMS CORPORATION, Otawara-shi (JP); KABUSHIKI KAISHA TOSHIBA, Minato-ku (JP)

(72) Inventor: Naoki YONEYAMA, Otawara-shi (JP)

(73) Assignees: TOSHIBA MEDICAL SYSTEMS CORPORATION, Otawara-shi (JP); KABUSHIKI KAISHA TOSHIBA, Minato-ku (JP)

(21) Appl. No.: 14/037,460

(22) Filed: Sep. 26, 2013

Related U.S. Application Data
(63) Continuation of application No. PCT/JP2013/066974, filed on Jun. 20, 2013.

(30) Foreign Application Priority Data
Jun. 29, 2012 (JP) 2012-147412

(51) Int. Cl. A61B 8/00; A61B 8/14

(52) U.S. Cl. CPC A61B 8/4254; A61B 8/4444; A61B 8/4477; A61B 8/14
USPC 600/443; 600/459

(57) ABSTRACT

According to one embodiment, an ultrasonic diagnostic apparatus includes a probe switching unit and a sensor selection unit. The switching unit switches an ultrasonic probe to be used for ultrasonic transmission/reception among a plurality of ultrasonic probes to which sensors configured to detect positions and directions in a predetermined space are respectively attached. The selection unit selects, when the switching unit performs switching between probes, a sensor attached to an ultrasonic probe to be used for ultrasonic transmission/reception from the respective sensors upon the switching based on a change in at least one of a position and a direction specified from a detection result from each of the sensors.

### CROSS-REFERENCE TO RELATED APPLICATIONS

This application is a Continuation Application of PCT Application No. PCT/JP2013/066974, filed Jun. 20, 2013 and based upon and claiming the benefit of priority from Japanese Patent Application No. 2012-147412, filed Jun. 29, 2012, the entire contents of all of which are incorporated herein by reference.

### FIELD

Embodiments described herein relate generally to an ultrasonic diagnostic apparatus which images the inside of the body of a patient with ultrasonic waves and a sensor selection apparatus.

### BACKGROUND

An ultrasonic diagnostic apparatus is an apparatus which acquires and displays an ultrasonic image of biological information. This apparatus is inexpensive and free from X-ray exposure as compared with other types of image diagnostic apparatuses such as X-ray diagnostic apparatuses and X-ray computerized tomography (CT) apparatuses, and is used as a useful apparatus for observing the inside of the body of a patient in real time.

An ultrasonic diagnostic apparatus is high in demand for applications making the most of the real-time performance of ultrasonic diagnosis such as a biopsy for definitive diagnosis in cancer screening and puncture guidance for radio-frequency ablation (RFA) treatment. Recently, in particular, there has been developed a technique of attaching a sensor for position and direction detection to an ultrasonic probe, displaying a CT image or magnetic resonance imaging (MRI) image including a morbid region as a reference image together with an ultrasonic image, and synchronously switching between the reference image and the ultrasonic image based on the position and direction detected by the sensor, thereby navigating an ultrasonic probe to the position of the morbid region.

When performing medical treatment using an ultrasonic diagnostic apparatus, the operator sometimes proceeds with operation while switching the probes to be used. For example, the operator uses a general-purpose ultrasonic probe to observe the inside of the body of a patient and uses an ultrasonic probe dedicated to puncture to perform puncturing operation.

Assume that only one sensor like that described above is prepared. In this case, when switching between ultrasonic probes, the operator needs to change the position of the sensor from the switching source probe to the switching destination probe. This is cumbersome operation. On the other hand, preparing sensors like those described above for the respective ultrasonic probes may cause erroneous recognition of the correspondence relationship between the sensors and the ultrasonic probes. This may lead to diagnosis errors.

Various kinds of problems arise in diagnosis, medical treatment, and the like executed upon attachment of a sensor for detecting a position and direction to an ultrasonic probe.

### BRIEF DESCRIPTION OF THE DRAWINGS

FIG. 1 is a block diagram showing the arrangement of the main part of an ultrasonic diagnostic apparatus (sensor selection apparatus) according to an embodiment;

FIG. 2 is a block diagram showing the arrangement of the main part of a positional information acquisition apparatus according to the above embodiment;

FIG. 3 is a perspective view showing an example of an attachment form of a magnetic sensor for the ultrasonic probe according to the above embodiment;

FIG. 4 is a view showing an example of a navigation window in the above embodiment;

FIG. 5 is a flowchart showing a procedure for the operation of the ultrasonic diagnostic apparatus according to the above embodiment;

FIG. 6 is a flowchart showing a procedure for sensor selection processing included in the flowchart of FIG. 5;

FIG. 7 is a view showing an example of a navigation window on which a histogram is placed according to the above embodiment; and

FIG. 8 is a view showing an example of a navigation window on which a histogram is placed according to the above embodiment.

### DETAILED DESCRIPTION

In general, according to one embodiment, an ultrasonic diagnostic apparatus consists of a probe switching unit and a sensor selection unit. The probe switching unit switches an ultrasonic probe to be used for ultrasonic transmission/reception among a plurality of ultrasonic probes to which sensors configured to detect positions and directions in a predetermined space are respectively attached. The sensor selection unit selects, when the probe switching unit performs switching between ultrasonic probes used for ultrasonic transmission/reception, a sensor attached to an ultrasonic probe to be used for ultrasonic transmission/reception from the respective sensors upon the switching based on a change in at least one of a position and a direction specified from a detection result from each of the sensors.

An embodiment will be described with reference to the accompanying drawings.

### [Ultrasonic Diagnostic Apparatus]

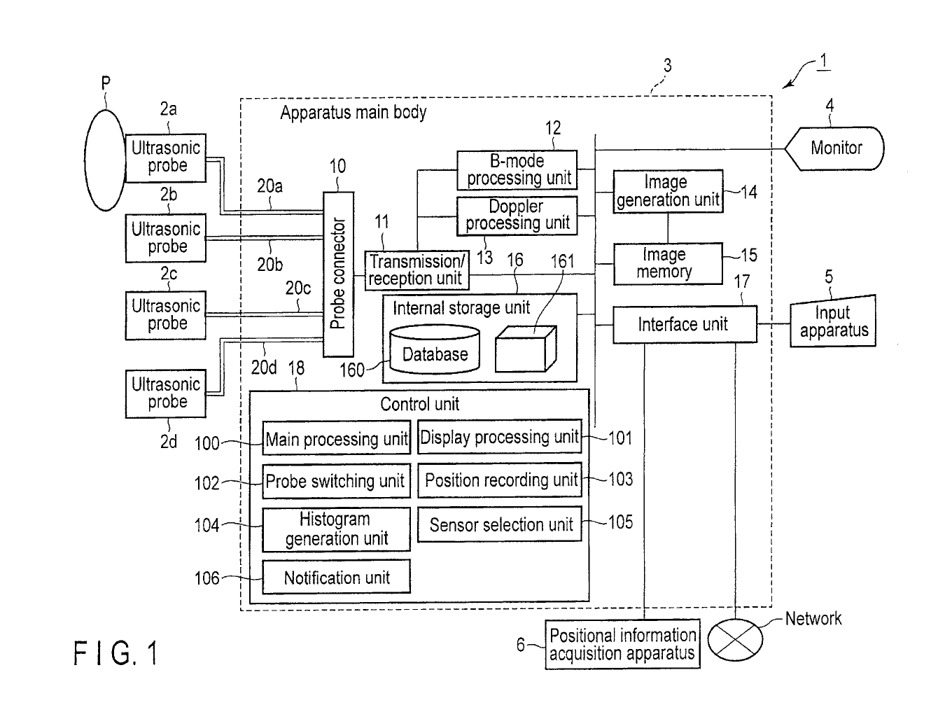

FIG. 1 is a block diagram showing the arrangement of the main part of an ultrasonic diagnostic apparatus 1 (sensor selection apparatus) according to an embodiment. As shown in FIG. 1, the ultrasonic diagnostic apparatus 1 according to this embodiment includes a plurality of ultrasonic probes 2, an apparatus main body 3, a monitor 4, an input apparatus 5, and a positional information acquisition apparatus 6. In the embodiment, in particular, the ultrasonic diagnostic apparatus 1 includes four ultrasonic probes 2a, 2b, 2c, and 2d. The number of ultrasonic probes of the ultrasonic diagnostic apparatus 1 is less than four or equal to or more than five. In the following description, ultrasonic probes 2a, 2b, 2c, and 2d will each be simply referred to as the ultrasonic probe 2 unless they are specifically discriminated from each other.

The ultrasonic probe 2 includes a plurality of piezoelectric transducers, a matching layer provided for each piezoelectric transducer, and a backing member which prevents ultrasonic waves from propagating backward from each piezoelectric transducer. Each piezoelectric transducer generates ultrasonic waves based on the driving signals supplied from the apparatus main body 3. Each piezoelectric transducer receives reflected waves from a patient P and outputs an electrical signal corresponding to the received reflected waves. The ultrasonic probe 2 may be any type of probe such as a sector type, linear type, or convex type probe.

When the ultrasonic probe 2 transmits ultrasonic waves to the patient P, the transmitted ultrasonic waves are sequentially reflected by a discontinuity surface of acoustic impedance of living tissue in the patient P. The plurality of piezoelectric transducers of the ultrasonic probe 2 receive the reflected ultrasonic waves as reflected wave signals. The amplitude of each received reflected wave signal depends on an acoustic impedance difference on the discontinuity surface by which ultrasonic waves are reflected. Each reflected wave signal produced when a transmitted ultrasonic pulse is reflected by a moving blood flow, the surface of the cardiac wall, or the like is subjected to a frequency shift depending on the velocity component of the moving body in the ultrasonic transmission direction due to the Doppler effect.

The input apparatus 5 includes a mouse, trackball, keyboard, and touch panel which are used to input, to the apparatus main body 3, various types of instructions, an instruction to set a region of interest (ROI), instructions concerning various types of image quality condition setting, and the like from the operator.

The monitor 4 displays a graphical user interface (GUI) for allowing the operator of the ultrasonic diagnostic apparatus 1 to input various types of instructions by using the input apparatus 5, and also displays the ultrasonic images generated by the apparatus main body 3.

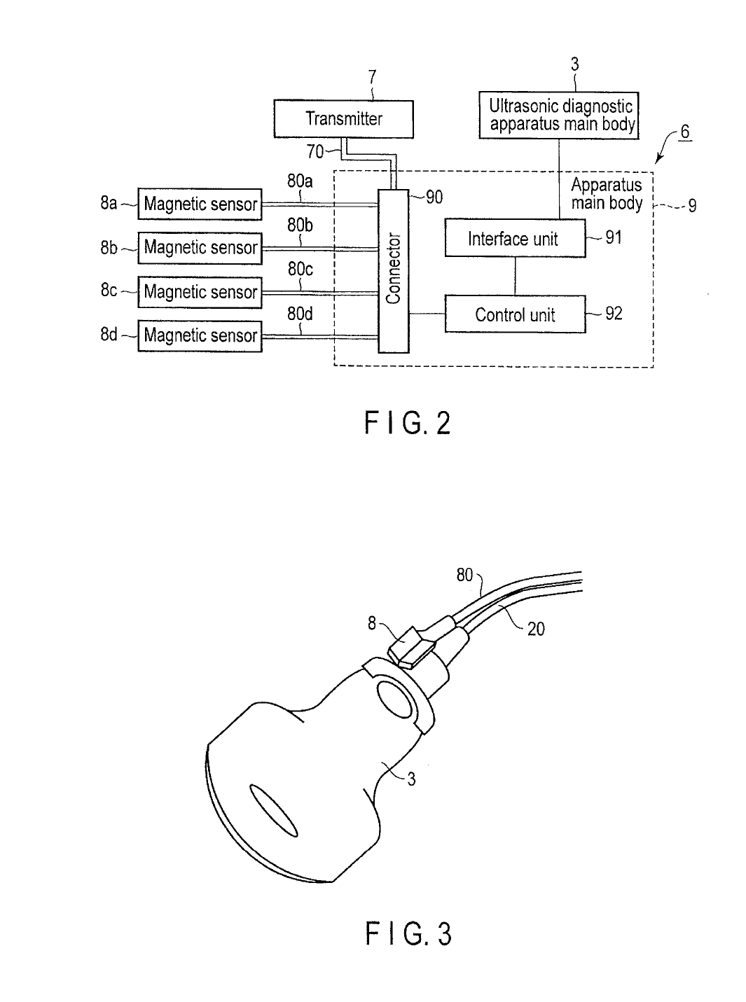

The positional information acquisition apparatus 6 acquires information concerning the position and direction of the ultrasonic probe 2 in a predetermined space. The detailed arrangement of the positional information acquisition apparatus 6 will be described later with reference to FIG. 2.

The apparatus main body 3 includes a probe connector 10, a transmission/reception unit 11, a B-mode processing unit 12, a Doppler processing unit 13, an image generation unit 14, an image memory 15, an internal storage unit 16, an interface unit 17, and a control unit 18.

The probe connector 10 connects a probe cable 20a which extends from ultrasonic probe 2a, a probe cable 20b which extends from ultrasonic probe 2b, a probe cable 20c which extends from ultrasonic probe 2c, and a probe cable 20d which extends from ultrasonic probe 2d. In the following description, probe cables 20a, 20b, 20c, and 20d will each be simply referred to as the probe cable 20 unless they are specifically discriminated from each other. The probe cable 20 is in charge of supplying power and transferring information between the apparatus main body 3 and the ultrasonic probe 2.

The transmission/reception unit 11 includes a trigger generation circuit, a delay circuit, and a pulser circuit as circuits belonging to the transmission system. The pulser circuit repeatedly generates rate pulses for the formation of transmission ultrasonic waves at a predetermined rate frequency. The delay circuit gives each rate pulse generated by the pulser circuit a delay time necessary to focus an ultrasonic wave generated by the ultrasonic probe 2 into a beam and determine transmission directivity for each channel. The trigger generation circuit supplies a driving signal (driving pulse) to the ultrasonic probe 2 at the timing based on this rate pulse. That is, the delay circuit arbitrarily adjusts the transmission direction from each piezoelectric transducer surface by changing the delay time given to each rate pulse.

The transmission/reception unit 11 includes an amplifier circuit, an analog-to-digital converter, and an adder as circuits belonging to the reception system. The amplifier circuit performs gain correction processing by amplifying the reflected wave signal received by the ultrasonic probe 2 for each channel. The analog-to-digital converter gives the gain-corrected reflected wave signals delay times necessary to determine reception directivities. The adder generates reflected wave data by performing addition processing for the reflected wave signals processed by the analog-to-digital converter. This addition processing performed by the adder enhances a reflection component from a direction corresponding to the reception directivity of the reflected wave signal.

The B-mode processing unit 12 receives reflected wave data from the transmission/reception unit 11, and performs logarithmic amplification, envelope detection processing, and the like for the received reflected wave data to generate data (B-mode data) whose signal intensity is expressed by a luminance level.

The Doppler processing unit 13 frequency-analyzes velocity information from the reflected wave data received from the transmission/reception unit 11 to extract a blood flow and tissue by the Doppler effect, and generates data (Doppler data) by extracting moving object information such as an average velocity, variance, and power at multiple points.

The image generation unit 14 generates ultrasonic image data from the B-mode data generated by the B-mode processing unit 12 and the Doppler data generated by the Doppler processing unit 13. More specifically, the image generation unit 14 converts a scanning line signal string for ultrasonic scanning into a scanning line signal string in a general video format typified by a TV format, thereby generating ultrasonic image data (B-mode image data or Doppler image data) for display from the B-mode data and the Doppler data.

The image memory 15 stores the ultrasonic image data generated by the image generation unit 14. The image memory 15 also stores an output signal (radio frequency [RF]) and image luminance signal immediately after passing through the transmission/reception unit 11, various types of raw data, image data acquired via the network, and the like, as needed. The data format of image data stored in the image memory 15 may be that after video format conversion displayed on the monitor 4 or that before coordinate conversion which is raw data generated by the B-mode processing unit 12 and the Doppler processing unit 13.

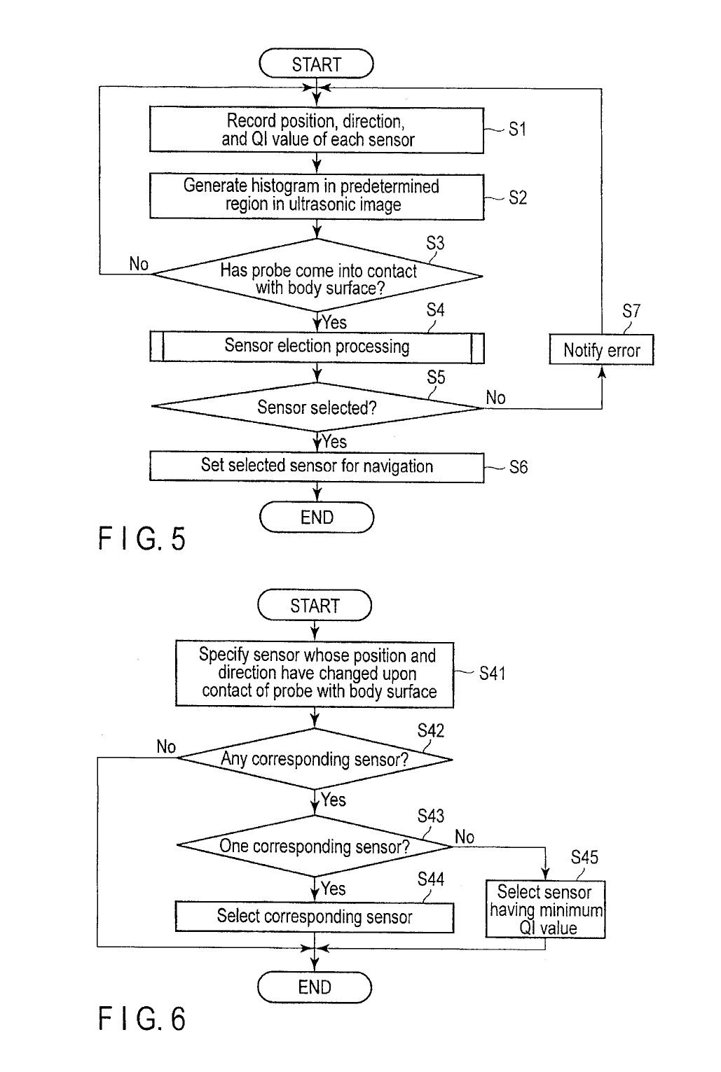

The internal storage unit 16 stores various types of data such as diagnostic information (for example, patient IDs and findings by doctors) and diagnostic protocols, a database 160, and volume data 161. The internal storage unit 16 is also used to, for example, archive the image data stored in the image memory 15, as needed. The application of the database 160 will be described later with reference to FIGS. 5 and 6. The volume data 161 concerns a predetermined region of the patient P, which is generated by another modality such as an X-ray CT apparatus or MRT apparatus.

The interface unit 17 controls transmission/reception of various types of information among the input apparatus 5, the positional information acquisition apparatus 6, a network or the like, and the apparatus main body 3.

The control unit 18 includes a central processing unit (CPU), a read-only memory (ROM), and a random access memory (RAM). The control unit 18 comprehensively controls the ultrasonic diagnostic apparatus 1. More specifically, the control unit 18 controls the operation of the transmission/reception unit 11, B-mode processing unit 12, Doppler processing unit 13, and image generation unit 14 based on various types of setting requests input by the operator via the input apparatus 5 and various types of control programs and various types of setting information read from the internal storage unit 16, the above ROM, or the like, and causes the monitor 4 to display the ultrasonic image data and the like stored in the image memory 15.

In this embodiment, in particular, the control unit 18 implements functions as a main processing unit 100, a display processing unit 101, a probe switching unit 102, a position recording unit 103, a histogram generation unit 104, a sensor selection unit 105, and a notification unit 106 by causing the above CPU to execute the control programs stored in the above ROM and or the like. These units will be described in detail below.

### [Positional Information Acquisition Apparatus]

The positional information acquisition apparatus 6 will be described in detail next.

FIG. 2 is a block diagram showing the arrangement of the main part of the positional information acquisition apparatus 6. The positional information acquisition apparatus 6 includes a transmitter 7 (magnetic field source), a plurality of magnetic sensors 8, and an apparatus main body 9. In this embodiment, in particular, the positional information acquisition apparatus 6 includes four magnetic sensors 8a, 8b, 8c, and 8d. The number of magnetic sensors 8 of the positional information acquisition apparatus 6 may be less than four or equal to more than five. In the following description, magnetic sensors 8a, 8b, 8c, and 8d will each be simply referred to as the magnetic sensor 8 unless they are specifically discriminated from each other.

The transmitter 7 forms a magnetic field centered on itself and extending to the outside.

The magnetic sensor 8 detects the strength and direction of the three-dimensional magnetic field formed by the transmitter 7, generates a signal corresponding to the detection result, and outputs it to the apparatus main body 9.

The apparatus main body 9 of the positional information acquisition apparatus 6 includes a connector 90, an interface unit 91, and a control unit 92.

The transmitter 7 is connected to the connector 90 via a transmitter cable 70. Magnetic sensor 8a is connected to the connector 90 via a sensor cable 80a. Magnetic sensor 8b is connected to the connector 90 via a sensor cable 80b. Magnetic sensor 8c is connected to the connector 90 via a sensor cable 80c. Magnetic sensor 8d is connected to the connector 90 via a sensor cable 80d. In the following description, sensor cables 80a, 80b, 80c, and 80d will each be simply referred to as the sensor cable 80 unless they are specifically discriminated from each other. The transmitter cable 70 is in charge of transferring information between the apparatus main body 9 and the transmitter 7. The sensor cable 80 is in charge of transferring information between the apparatus main body 9 and the magnetic sensors 8.

The interface unit 91 controls transmission/reception of various types of information between the apparatus main body 3 of the ultrasonic diagnostic apparatus 1 and the apparatus main body 9 of the positional information acquisition apparatus 6.

The control unit 92 includes a CPU, a ROM, and a RAM and comprehensively controls the positional information acquisition apparatus 6.

The magnetic sensor 8 is attached to or detached from the ultrasonic probe 2. FIG. 3 shows an example of a form of attaching the magnetic sensor 8 to the ultrasonic probe 2. In this example, the magnetic sensor 8 is attached to the base portion of the probe cable 20 connected to the ultrasonic probe 2 through a detachable mechanism having an arbitrary shape. Note however that the position at which the magnetic sensor 8 is attached to the ultrasonic probe 2 may be another position.

The control unit 92 calculates a position (x, y, z) and direction (θx, θy, θz) of the magnetic sensor 8 in a three-dimensional coordinate space (X, Y, Z) defined by the X-, Y-, and Z-axes, with, for example, the transmitter 7 being the origin, based on the signal received from the magnetic sensor 8. In this case, let θx be the rotational angle of the magnetic sensor 8 centered on the X-axis, θy be the rotational angle of the magnetic sensor 8 centered on the Y-axis, and θz be the rotational angle of the magnetic sensor 8 centered on the Z-axis.

The control unit 92 calculates a quality index (QI) value representing a magnetic field disturbance strength (the change frequency of magnetic field per unit time) at the position of the magnetic sensor 8. The control unit 92 calculates this QI value by substituting the change count or change amount of the position (x, y, z) and direction (θx, θy, θz) per unit time into a predetermined mathematical expression. Assume that in this embodiment, this mathematical expression is defined to increase the QI value with an increase in magnetic field disturbance, decrease the QI value with a decrease in magnetic field disturbance, and make the QI value reflect a low-frequency change in position (x, y, z) and direction (θx, θy, θz) than a high-frequency change in them. The above low-frequency change occurs when the operator moves the magnetic sensor 8 with his/her hand. The above high-frequency change originates from, for example, magnetic field disturbance or the like due to a metal existing near the magnetic sensor 8.

The control unit 92 outputs the calculated position (x, y, z), direction (θx, θy, θz), and QI value to the apparatus main body 3 of the ultrasonic diagnostic apparatus 1 via the interface unit 91.

As described above, ultrasonic probes 2a to 2d are connected to the probe connector 10 and magnetic sensors 8a to 8d are connected to the connector 90. Namely, the means of connection of ultrasonic probes 2a to 2d and the means of connection of magnetic sensors 8a to 8d are different from each other. Consequently, ultrasonic probes 2a to 2d and magnetic sensors 8a to 8d may be independently controlled to switch.

### [Navigation Function]

A form of diagnosis and medical treatment executed by using the ultrasonic diagnostic apparatus 1 having the above arrangement will be described.

The ultrasonic diagnostic apparatus 1 has a navigation function of guiding the position and direction of the ultrasonic probe 2 to a target such as a tumor found in diagnosis using the volume data 161 by displaying an ultrasonic image and a reference image in the same window and switching the reference image in synchronism with a change in ultrasonic image in accordance with the operation of the ultrasonic probe 2. In this embodiment, the above ultrasonic image is the B-mode image obtained by transmission/reception of ultrasonic waves using the ultrasonic probe 2. The above two-dimensional image is the one generated from the volume data 161 obtained by another modality.

The main processing unit 100 executes the main processing associated with the navigation function. In addition, the display processing unit 101, the probe switching unit 102, the position recording unit 103, the histogram generation unit 104, the sensor selection unit 105, and the notification unit 106 execute auxiliary processing.

When using the navigation function, the operator attaches the magnetic sensor 8 to the ultrasonic probe 2 to be used for transmission/reception of ultrasonic waves (to be referred to as the use target ultrasonic probe 2 hereinafter) and places the transmitter 7 near the range of use of the ultrasonic probe 2 so as to guarantee the accurate detection of the position (x, y, z) and direction (θx, θy, θz). As preparatory work for navigation, the operator sets a target such as a tumor, aligns the volume data 161 with the axis (angle) of the ultrasonic probe 2, and performs mark alignment of aligning one point among the coordinates after axis alignment with another corresponding point. Assume that in this embodiment, when the operator performs this preparatory work, a combination of the use target ultrasonic probe 2 and the magnetic sensor 8 attached to the ultrasonic probe 2 is known.

When setting a target, the operator designates the central position and range of a tumor or the like included in the volume data 161 by operating the input apparatus 5. When the operator performs this operation, the main processing unit 100 performs the processing for setting the coordinates of the designated central position and range to the target.

When performing axis alignment, the operator places the use target ultrasonic probe 2 in a direction perpendicular to the body axis of the patient P (in the axial plane direction) and issues an instruction to execute axis alignment by operating the input apparatus 5. In accordance with this instruction, the main processing unit 100 performs the processing for aligning the three axes, i.e., the X-, Y-, and Z-axes of the magnetic sensor 8 attached to the ultrasonic probe 2 with the three axes of the volume data 161.

When performing mark alignment, the operator can operate the input apparatus 5 to designate the feature portion depicted on both the ultrasonic image obtained by causing the use target ultrasonic probe 2 to transmit and receive ultrasonic waves and the reference image generated by the volume data 161. In accordance with this designation, the main processing unit 100 performs the processing for associating the volume data 161 with the coordinates of the magnetic sensor 8.

Through the above preparation processing, it is possible to generate a reference image concerning the same slice as that of the ultrasonic image obtained by the ultrasonic probe 2 from the volume data 161 and navigate the use target ultrasonic probe 2 to the target.

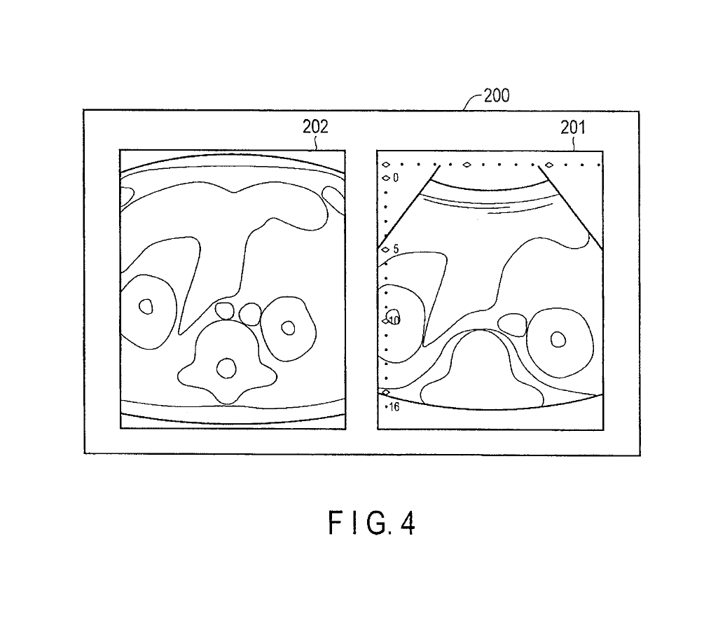

After the completion of the preparation processing, the display processing unit 101 causes the monitor 4 to display a navigation window 200 including a real-time ultrasonic image 201 and a reference image 202 generated from the volume data 161, as shown in, for example, FIG. 4.

Every time the image generation unit 14 generates ultrasonic image data concerning the use target ultrasonic probe 2 and the image memory 15 stores the ultrasonic image data, the display processing unit 101 replaces the ultrasonic image 201 with the image based on the ultrasonic image data. With this operation, the ultrasonic image 201 depicting the inside of the body of the patient P is displayed in real time. The display processing unit 101 generates the two-dimensional image data of a slice corresponding to the ultrasonic image 201 from the volume data 161 based on the position (x, y, z) and direction (θx, θy, θz) of the magnetic sensor 8 attached to the use target ultrasonic probe 2, and replaces the reference image 202 with the image based on this two-dimensional image data. This synchronizes the ultrasonic image 201 with the reference image 202. In addition, when a target is set on the ultrasonic image 201 and the reference image 202, the display processing unit 101 emphasizes the target by adding markers on the two images 201 and 202.

### [Switching Between Ultrasonic Probes and Selection of Magnetic Sensor]

It is sometimes necessary to switch the use target ultrasonic probe 2 among ultrasonic probes 2a to 2d during diagnosis or medical treatment using the above navigation. When, for example, performing RFA treatment, the operator generally performs the above preparatory work by using a large-diameter convex probe configured to obtain an image depicting with a high resolution and high contrast from a shallow portion to a deep portion and performs subsequent puncturing operation by using a small-diameter convex probe.

When switching the use target ultrasonic probe 2, the operator designates a probe to be used after switching from ultrasonic probes 2a to 2d by operating the input apparatus 5. The operator may perform this designating operation by operating buttons corresponding to the respective connecting terminals of the probe connector 10 which are provided on the input apparatus 5.

When the operator designates the ultrasonic probe 2 to be used after switching, the probe switching unit 102 issues an instruction to the transmission/reception unit 11 to set the designated ultrasonic probe as the ultrasonic probe 2 as an input/output destination of driving pulses and reflected wave signals. Upon receiving this instruction, the transmission/reception unit 11 generates ultrasonic waves by sequentially supplying driving pulses to the designated ultrasonic probe 2 and generates reflected wave data by sequentially receiving reflected wave signals from the ultrasonic probe 2. Ultrasonic image data based on the reflected wave data is generated through the B-mode processing unit 12 and the image generation unit 14, and is stored in the image memory 15. Therefore, the ultrasonic image 201 displayed on the navigation window 200 is also the image obtained by using the ultrasonic probe 2 after the switching operation.

Upon switching the use target ultrasonic probe 2, the ultrasonic diagnostic apparatus 1 needs to recognize the magnetic sensor 8 attached to the ultrasonic probe 2 after switching. In this embodiment, when switching the use target ultrasonic probe 2, the control unit 18, more specifically, the position recording unit 103, the histogram generation unit 104, the sensor selection unit 105, and the notification unit 106 execute the processing shown in the flowcharts of FIGS. 5 and 6 to automate a procedure associated with this recognition.

These flowcharts will be described below. Assume that the positional information acquisition apparatus 6 periodically calculates the position (x, y, z), direction (θx, θy, θz), and QI value of each of magnetic sensors 8a to 8d and transmits them to the apparatus main body 3 of the ultrasonic diagnostic apparatus 1.

In the flowchart of FIG. 5, first of all, the position recording unit 103 writes, in the database 160, the positions (x, y, z) and directions (θx, θy, θz) of magnetic sensors 8a to 8d transmitted from the positional information acquisition apparatus 6 to the interface unit 17, and QI values (step S1).

Subsequently, the histogram generation unit 104 generates the histogram data of pixels included in a predetermined region defined in the currently displayed ultrasonic image 201 (step S2). This histogram data is data representing the distribution of the numbers of pixels for each luminance. The above predetermined region is, for example, a region defined in advance near the focus point of ultrasonic transmission or near the central portion of a displayed image or a region such as an ROI arbitrarily set by the operator.

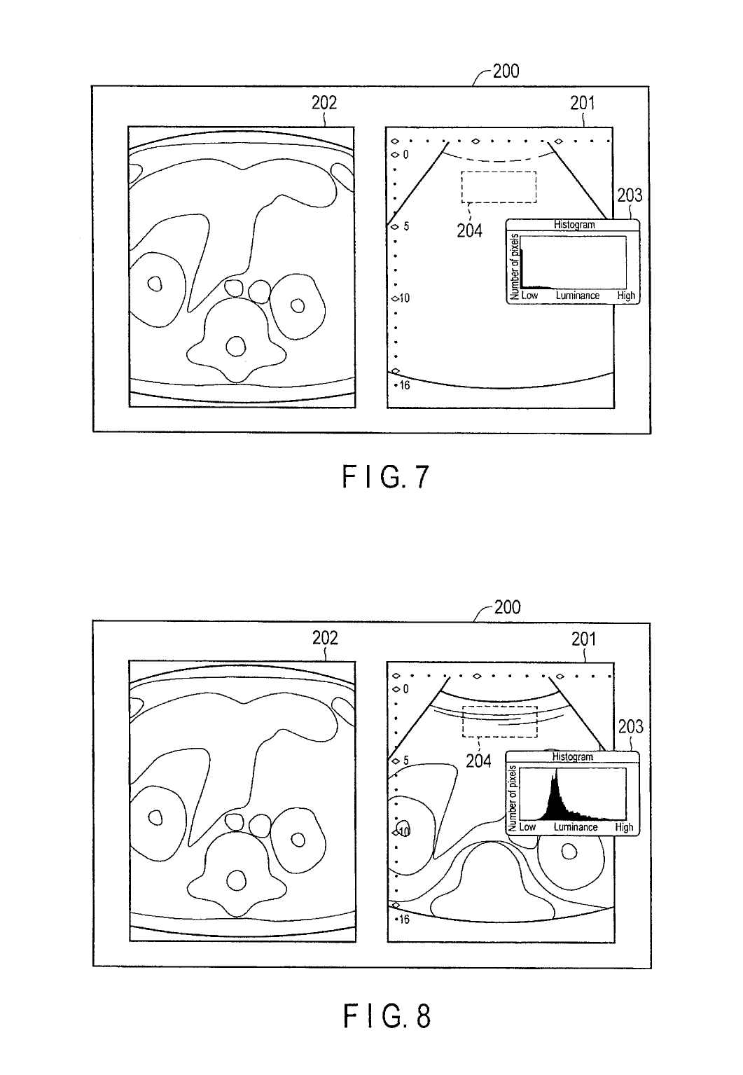

The histogram based on the histogram data generated in step S2 may be displayed on the navigation window 200. FIGS. 7 and 8 each show an example of the navigation window 200 displaying a histogram 203 based on histogram data. A rectangular broken line 204 in each of FIGS. 7 and 8 represents the above predetermined region. The abscissa and ordinate of the histogram 203 respectively represent luminance and number of pixels. The navigation window 200 shown in FIG. 7 is an example of the window displayed while the use target ultrasonic probe 2 is not placed near the body surface of the patient P. In this case, since almost no reflected wave is obtained from the inside of the field of view, the ultrasonic image 201 becomes an image having the lowest luminance as a whole. Therefore, the histogram 203 hardly shows the distribution of the numbers of pixels. In contrast, the navigation window 200 shown in FIG. 8 is an example of the window displayed while the use target ultrasonic probe 2 is in contact with the body surface of the patient P. In this case, since proper reflected waves can be obtained from the inside of the field of view, the ultrasonic image 201 becomes an image having high luminance as a whole. Therefore, the histogram 203 shows the clear distribution of the numbers of pixels.

After step S2, the histogram generation unit 104 determines, based on the histogram data currently generated in step S2 and the histogram data previously generated in step S2, whether the use target ultrasonic probe 2 which has not been in contact with the body surface of the patient P has moved to come into contact with the body surface of the patient P (step S3). Note that no previous histogram data exists at the start of the processing shown in the flowchart of FIG. 5. In this case, the histogram generation unit 104 determines whether the use target ultrasonic probe 2 has not moved to come into contact with the body surface of the patient P (NO in step S3), and executes steps S1 and S2 again.

It is possible to perform the determination in step S3 by using, for example, the average luminance value obtained from histogram data. That is, the histogram generation unit 104 calculates an average luminance value B1 of the currently generated histogram data and an average luminance value B2 of the previously generated histogram data, and compares a difference ΔB(=B1−B2) between them and a predetermined threshold Bs. The threshold Bs is used to determine whether the use target ultrasonic probe 2 which is not in contact with the body surface of the patient P has come into contact with or come close to the body surface of the patient P. The concrete value of the threshold Bs may be determined experimentally, empirically, or theoretically. If the comparison result indicates that ΔB≦Bs, the histogram generation unit 104 determines that the use target ultrasonic probe 2 has not moved to come into contact with the body surface of the patient P (No in step S3). In this case, the histogram generation unit 104 executes steps S1 and S2 again.

If ΔB>Bs, the histogram generation unit 104 determines that the operator has moved the use target ultrasonic probe 2 to bring it into contact with the body surface of the patient P (YES in step S3). In this case, the sensor selection unit 105 executes sensor selection processing (step S4).

In sensor selection processing, the sensor selection unit 105 operates in accordance with the flowchart of FIG. 6. When the use target ultrasonic probe 2 comes into contact with the body surface of the patient P, the sensor selection unit 105 specifies the magnetic sensor 8 whose at least one of the position (x, y, z) and the direction (θx, θy, θz) has changed (step S41). More specifically, the sensor selection unit 105 refers to the database 160 to specify the magnetic sensor 8 whose position (x, y, z) has changed to a predetermined distance Ds or more or whose at least one of angles θx, θy, and θz has changed to a predetermined angle Os or more at almost the time of ultrasonic transmission/reception to obtain ultrasonic image data as the generation source of histogram data in immediately preceding step S2. The distance Ds and the angle θs both are thresholds to determine whether the magnetic sensor 8 has moved together with the use target ultrasonic probe 2. The concrete values of the distance Ds and angle θs may be determined experimentally, empirically, or theoretically.

Subsequently, the sensor selection unit 105 determines whether at least one of the magnetic sensors 8 has been able to be specified in step S41 (step S42). If at least one magnetic sensor 8 has been able to be specified (YES in step S42), the sensor selection unit 105 determines whether only one magnetic sensor 8 has been able to be specified (step S43). If only one magnetic sensor 8 has been able to be specified (YES in step S43), the sensor selection unit 105 selects the magnetic sensor 8 as the magnetic sensor attached to the use target ultrasonic probe 2 (step S44).

If there are a plurality of magnetic sensors 8 which have been able to be specified in step S41 (NO in step S43), the sensor selection unit 105 selects one of the magnetic sensors 8 (step S45). More specifically, the sensor selection unit 105 refers to the database 160 to select the magnetic sensor 8 whose QI value is minimum (most preferable) at almost the time of ultrasonic transmission/reception to obtain ultrasonic image data as the generation source of the histogram data in immediately preceding step S2 as the magnetic sensor attached to the use target ultrasonic probe 2. As a QI value, it is possible to use a value at one specific time point or an average value in a predetermined period of time.

With step S44 or S45, the sensor selection unit 105 terminates the sensor selection processing. If the sensor selection unit 105 could not specify any of the magnetic sensors 8 in step S41 (NO in step S42), the sensor selection unit 105 terminates the sensor selection processing without selecting any magnetic sensor 8.

After the sensor selection processing, as indicated by the flowchart of FIG. 5, the main processing unit 100 determines whether the magnetic sensor 8 attached to the use target ultrasonic probe 2 has been selected in the sensor selection processing (step S5). If the sensor selection processing has been complete through step S44 or S45, the main processing unit 100 determines that the magnetic sensor 8 has been selected (YES in step S5), and sets the selected magnetic sensor 8 for subsequent navigation (step S6). With step S6, the processing shown in the flowchart of FIG. 5 is complete. Subsequently, the display processing unit 101 generates the two-dimensional image data of a slice corresponding to the ultrasonic image 201 from the volume data 161 based on the position (x, y, z) and direction (θx, θy, θz) of the magnetic sensor 8 set in step S6, and replaces the reference image 202 with the image based on the two-dimensional image data.

If no magnetic sensor 8 has been selected in the sensor selection processing (NO in step S5), the notification unit 106 notifies an error (step S7). The notification unit 106 may perform this notification by, for example, displaying, on the monitor 4, a message stating that the magnetic sensor 8 is not properly attached to the use target ultrasonic probe 2. After this notification, the process returns to step S1. At this time, when the operator separates the use target ultrasonic probe 2 from the body surface of the patient P, attaches any one of the magnetic sensors 8, and brings the ultrasonic probe 2 into contact with the body surface of the patient P again, the magnetic sensor 8 is selected by sensor selection processing. The selected magnetic sensor 8 is set for navigation, and the processing shown in the flowchart of FIG. 5 is complete.

As described above, when the operator switches the use target ultrasonic probe 2 among ultrasonic probes 2a to 2d, the ultrasonic diagnostic apparatus 1 selects the magnetic sensor attached to the ultrasonic probe 2 newly set as a use target by the above switching operation based on a change in at least one of the position (x, y, z) and direction (θx, θy, θz) specified from outputs from magnetic sensors 8a to 8d. This arrangement makes it unnecessary for the operator to designate the magnetic sensor 8 attached to the ultrasonic probe 2 upon switching of the use target ultrasonic probe 2. When the operator is to designate the magnetic sensor 8, there is a danger that the operator may erroneously determine the correspondence relationship between the ultrasonic probe 2 and the magnetic sensor 8 to lead to a diagnosis error. The arrangement of this embodiment is free from such a situation.

More specifically, the ultrasonic diagnostic apparatus 1 selects a sensor, of magnetic sensors 8a to 8d, whose change in at least one of the direction (x, y, z) and the direction (θx, θy, θz) specified from outputs from the respective magnetic sensors exceeds a predetermined threshold as a sensor attached to the use target ultrasonic probe 2. If there are a plurality of sensors whose changes in at least one of the direction (x, y, z) and the direction (θx, θy, θz) exceed the above threshold, the ultrasonic diagnostic apparatus 1 selects a selector whose QI value calculated from an output from it is minimum as a sensor attached to the use target ultrasonic probe 2. In general, the transmitter 7 and its peripheral devices are arranged so as to make the magnetic sensor 8 attached to the ultrasonic probe 2 have good magnetic field detection sensitivity while the use target ultrasonic probe 2 is in contact with the body surface of the patient P. For this reason, while the use target ultrasonic probe 2 is in contact with the body surface of the patient P, the QI value concerning the magnetic sensor 8 attached to the ultrasonic probe 2 is estimated to exhibit a good value (small value). Considering the QI value as in this embodiment can therefore accurately select the magnetic sensor 8 attached to the use target ultrasonic probe 2.

The ultrasonic diagnostic apparatus 1 selects, as the sensor attached to the use target ultrasonic probe 2, the magnetic sensor 8 whose at least one of the position (x, y, z) and the direction (θx, θy, θz) has changed when a histogram concerning the luminances of pixels included in a predetermined region in the ultrasonic image 201 undergoes a predetermined change (a change in average luminance value exceeding the threshold Bs). When the histogram changes in this manner, it is assumed that the operator has moved the use target ultrasonic probe 2 to bring it into contact with the body surface of the patient P. At this timing, the magnetic sensor 8 attached to the ultrasonic probe 2 should have moved. Therefore, considering a change in the position (x, y, z) and direction (θx, θy, θz) of each of magnetic sensors 8a to 8d at the same timing will improve the accuracy of the selection of the magnetic sensor 8.

When failing to select the magnetic sensor 8 attached to the use target ultrasonic probe 2, the ultrasonic diagnostic apparatus 1 further notifies an error. This notification can make the operator notice that, for example, he/she has forgotten to attach the magnetic sensor 8 to the use target ultrasonic probe 2.

In this manner, the arrangement according to this embodiment can greatly improve the convenience in diagnosis, medical treatment, or the like executed upon attaching the magnetic sensor 8 to the ultrasonic probe 2.

### [Modification]

Note that the arrangement disclosed in the above embodiment can be variously modified and executed.

For example, the above embodiment has exemplified the use of the magnetic sensor 8 as a sensor for detecting the position (x, y, z) and the direction (θx, θy, θz). However, it is possible to use a sensor configured to detect a physical quantity other than a magnetic field representing the position (x, y, z) and the direction (θx, θy, θz) instead of the magnetic sensor 8. It is possible to use, as such a sensor, an optical sensor which outputs a signal corresponding to received light.

In addition, the above embodiment has exemplified the case in which the apparatus performs the determination in step S3 by using the average luminance value obtained from histogram data. However, the apparatus may perform the determination in step S3 by using a parameter other than an average luminance value. It is possible to use, as such a parameter, for example, the variance value obtained from histogram data.

Furthermore, in the above embodiment, the apparatus generates the image data of the reference image 202 from the volume data 161 based on the position (x, y, z) of the magnetic sensor 8 attached to the use target ultrasonic probe 2. However, since it is also assumed that the attachment position of the magnetic sensor 8 on the ultrasonic probe 2 changes depending on the shape or the like of the ultrasonic probe 2, if the attachment position of the magnetic sensor 8 on the ultrasonic probe 2 changes before and after switching operation, the position (x, y, z) changes before and after the switching operation. In this case, it is necessary to execute preparatory work for mark alignment or the like again. In consideration of this point, it is possible to eliminate the necessity to perform preparatory work again by correcting the position (x, y, z) used for navigation by considering the attachment positions and the like of the magnetic sensor 8 on the ultrasonic probe 2 before and after switching operation.

The control unit 92 which the apparatus main body 9 of the positional information acquisition apparatus 6 has may implement some or all of the main processing unit 100, the display processing unit 101, the probe switching unit 102, the position recording unit 103, the histogram generation unit 104, the sensor selection unit 105, and the notification unit 106 which are implemented by the control unit 18 of the apparatus main body 3 of the ultrasonic diagnostic apparatus 1 in the above embodiment. When the control unit 92 implements elements including the sensor selection unit 105, in particular, the positional information acquisition apparatus 6 functions as a sensor selection apparatus.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a probe switching unit configured to switch an ultrasonic probe to be used for ultrasonic transmission/reception among a plurality of ultrasonic probes to which sensors configured to detect positions and directions in a predetermined space are respectively attached; and
a sensor selection unit configured to select, when the probe switching unit performs switching between ultrasonic probes used for ultrasonic transmission/reception, a sensor attached to an ultrasonic probe to be used for ultrasonic transmission/reception from the respective sensors upon the switching based on a change in at least one of a position and a direction specified from a detection result from each of the sensors.

2. The ultrasonic diagnostic apparatus of claim 1, wherein each of the sensors detects a magnetic field generated from a magnetic field source as the physical quantity, and
the sensor selection unit selects a sensor, of the respective sensors, whose at least one of a position and a direction specified from a detection result from each of the respective sensors exceeds a predetermined threshold as a sensor attached to an ultrasonic probe to be used for ultrasonic transmission/reception upon switching by the probe switching unit, and if there are a plurality of sensors whose change in at least one of the position and the direction exceeds the threshold, selects a sensor whose index indicating a magnetic field disturbance derived from a detection result from each of the sensors is optimal as a sensor attached to an ultrasonic probe to be used for ultrasonic transmission/reception upon the switching.

3. The ultrasonic diagnostic apparatus of claim 1, further comprising a histogram generation unit configured to generate a histogram concerning luminances of pixels included in an ultrasonic image obtained by using an ultrasonic probe to be used for ultrasonic transmission/reception upon switching by the probe switching unit,
wherein when a histogram generated by the histogram generation unit undergoes a predetermined change, the sensor selection unit selects a sensor whose at least one of a position and a direction specified by a detection result has changed as a sensor attached to an ultrasonic probe to be used for ultrasonic transmission/reception upon switching by the probe switching unit.

4. The ultrasonic diagnostic apparatus of claim 3, wherein the histogram generation unit generates a histogram concerning luminances of pixels included in any one of a region set near a focus point of ultrasonic transmission by an ultrasonic probe to be used for ultrasonic transmission/reception upon switching by the probe switching unit, a region set near a central portion of the ultrasonic image, and an arbitrary region designated by an operator.

5. The ultrasonic diagnostic apparatus of claim 1, further comprising a notification unit configured to notify an error when the sensor selection unit is not configured to select any sensor.

6. A sensor selection apparatus comprising a sensor selection unit configured to select, when an ultrasonic probe to be used for ultrasonic transmission/reception is switched among a plurality of ultrasonic probes to which sensors configured to detect positions and directions in a predetermined space are respectively attached, a sensor attached to an ultrasonic probe to be used for ultrasonic transmission/reception upon the switching from the sensors based on a change in at least one of a position and a direction specified from a detection result from each of the sensors.

7. The sensor selection apparatus of claim 6, wherein each of the sensors detects a magnetic field generated from a magnetic field source as the physical quantity, and
the sensor selection unit selects a sensor, of the respective sensors, whose at least one of a position and a direction specified from a detection result from each of the respective sensors exceeds a predetermined threshold as a sensor attached to an ultrasonic probe to be used for ultrasonic transmission/reception upon the switching, and if there are a plurality of sensors whose change in at least one of the position and the direction exceeds the threshold, selects a sensor whose index indicating a magnetic field disturbance derived from a detection result from each of the sensors is optimal as a sensor attached to an ultrasonic probe to be used for ultrasonic transmission/reception upon switching.

8. The sensor selection apparatus of claim 6, further comprising a histogram generation unit configured to generate a histogram concerning luminances of pixels included in an ultrasonic image obtained by using an ultrasonic probe to be used for ultrasonic transmission/reception upon the switching,
wherein when a histogram generated by the histogram generation unit undergoes a predetermined change, the sensor selection unit selects a sensor whose at least one of a position and a direction specified by a detection result has changed as a sensor attached to an ultrasonic probe to be used for ultrasonic transmission/reception upon switching.

9. The sensor selection apparatus of claim 6, wherein the histogram generation unit generates a histogram concerning luminances of pixels included in any one of a region set near a focus point of ultrasonic transmission by an ultrasonic probe to be used for ultrasonic transmission/reception upon the switching, a region set near a central portion of the ultrasonic image, and an arbitrary region designated by an operator.

10. The sensor selection apparatus of claim 6, further comprising a notification unit configured to notify an error when the sensor selection unit is not configured to select any sensor.
